(19) United States
(12) Plant Patent Application Publication
(10) Pub. No.: US 2003/0070198 P1
(43) Pub. Date: Apr. 10, 2003

(54) OSTEOSPERMUM PLANT NAMED 'KAKEGAWA AU7'

(76) Inventor: Masao Kanno, Chigasaki- City (JP)

Correspondence Address: JONDLE & ASSOCIATES P.C., 9085 EAST MINERAL CIRCLE, SUITE 200, CENTENNIAL, CO, 80112, US

(21) Appl. No.: 09/971,628

(22) Filed: Oct. 9, 2001

(51) Int. Cl. A01H 5/00

(52) U.S. Cl. PLT/360

(57) ABSTRACT

An Osteospermum cultivar particularly distinguished by its white and pink flower color and tendency to hold flowers open later in the evening.

### BACKGROUND OF THE NEW PLANT

The present invention originated in 1984 by a cross between breeding line No. 8 (unpatented), possessing white flowers and breeding line No.16 (unpatented), possessing white flowers and a blue eye. The F₁ seed from this cross was sown in Fall, 1984 and in Spring, 1985 two selections were made and the two selected plants were intercrossed. The F₁ seed from these two sister lines was sown in Fall, 1985 and in Spring, 1986 six plants were selected for intercrossing. The F₁ seed from these six plants was again sown in Fall, 1986. In 1987 a pink flowered selection named No.131 (unpatented) was made. In 1988 breeding line No. 99 (unpatented) was crossed to No. 131. In 1989 the F₁ plant No. 209 (unpatented) was selected for its pale pink flowers and dwarf habit. In 1990, line No. 209 was crossed to line No.159 (unpatented). In 1991 two F₁ plants were selected from this cross for their light pink flower color and tendency to hold flowers open later in the evening and named No. 2127 (unpatented) and No. 237 (unpatented). In 1992 these two plants were crossed to produce F₂ seed. In 1993 the F₂ plant, G7-683, was selected from the F₂ population for its flower color and holding flowers open later. In Fall, 1993 the plant was vegetatively propagated and stability was confirmed in Spring, 1994. The line was established as ‘Kakegawa AU7’, and determined to have its characteristics firmly fixed.

### DESCRIPTION OF PHOTOGRAPH

This new Osteospermum plant is illustrated by the accompanying photograph which shows blooms, and foliage of the plant in full color, the colors shown being as true as can be reasonably obtained by conventional photographic procedures.

FIG. 1 shows a view of three plants propagated in a 20 cm diameter pot;

FIG. 2 shows a close-up view of a single inflorescence.

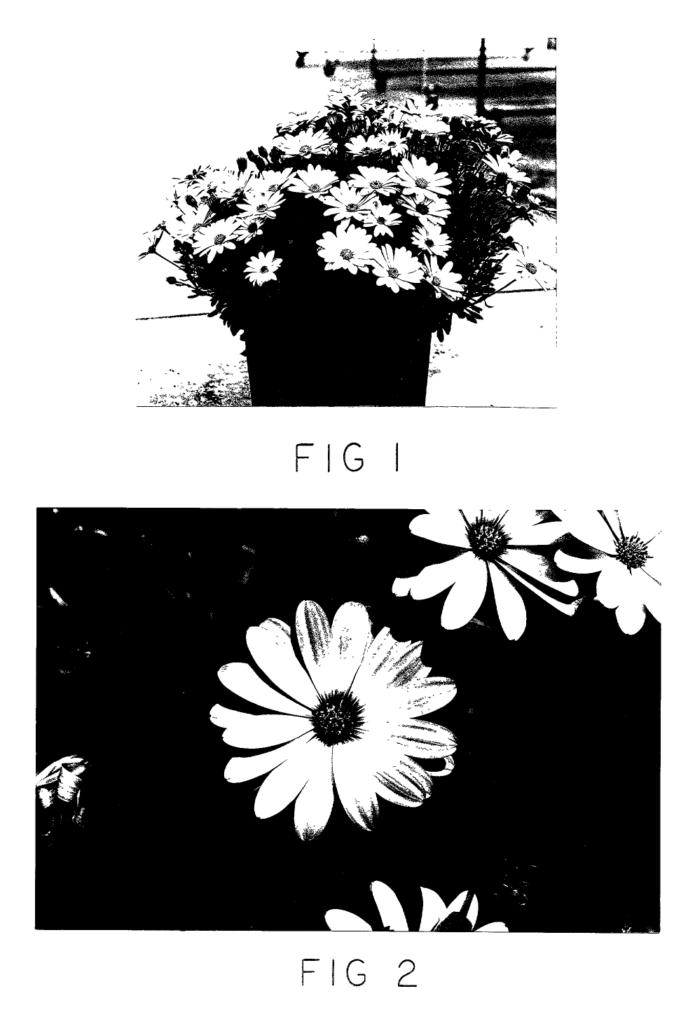

### DESCRIPTION OF THE NEW CULTIVAR

The following detailed descriptions set forth the distinctive characteristics of ‘Kakegawa AU7’. The data which defines these characteristics were collected from asexual reproductions carried out in Salinas, Calif. Data was collected on plants 28 weeks from transplanting rooted cuttings into 16 cm diameter pots and growing as described below. Color references are to the RHS Colour Chart of The Royal Horticultural Society of London (RHS).

### DESCRIPTION OF THE NEW PLANT

Classification: Family: Compositae Botanical: Osteospermum fruticosum Commercial: Cape Daisy

Parentage: Female parent—breeding line No.2127 (unpatented) Male parent—breeding line No. 237 (unpatented)

Growth:

Time to produce a rooted cutting—Cuttings will colonize a 2.5 cm diameter by 2.5 cm tall greenhouse tray cell with peat-based plant media in approximately five weeks. Cuttings are dipped in a normal dilution (1:9) of Dip ‘N Grow™ root inducing solution in water. The trays are misted hourly during rooting.

Environmental conditions for plant growth—Rooted cuttings are transplanted to pots with a 16 cm diameter, one plant per pot. Peat moss-based growing media is used. The pots are watered using a 150-200 ppm fertilizer solution containing 18% nitrogen, 8% phosphorus and 18% potassium. The soil is allowed to dry between waterings. During the first few weeks after transplanting the plants should have evening temperatures around 15-18° C. for good root growth. When plants reach 7.5-10 cm in height they are pinched back to 5-6 leaves to promote branching. The plants are grown through the winter in cool greenhouses (10-15° C.) with no shading to keep their habit compact. In spring, after the plants have received at least four weeks of cool conditions to vernalize, warmer temperatures can be used to promote blooming. Spring and summer daytime high temperatures in Salinas, California, where the data was collected, range from 16-25° C.

Time to bloom from propagation—18-20 weeks when rooted vegetative cuttings are transferred to a 16 cm diameter pot in late Fall and given several weeks of below 50° F. temperature prior to increasingly warmer spring weather.

Habit.—vigorous, well branched

Life cycle.—perennial

Plant:

Form.—upright

Plant Size.—height is 35-40 cm; width is 35-40 cm

Stems:

Internode length.—0.8-1.0 cm

Color.—Yellow-green (RHS N144A)

Description.—Strong, erect, herbaceous

Stem diameter.—2.0-4.0 mm

Pubescence.—Short, transparent

Leaves:

Arrangement.—alternate

Shape.—spatulate

Apex.—mucronate

Base.—oblique, sessile

Margin.—serrate

Venation.—pinnate

Color.—Upper is green (RHS 137A); Lower is green (RHS 137C)

Inflorescence:

Type.—Capitulum (head); disk flowers are staminate and ray flowers are carpellate.

Diameter.—5.5-6.0 cm

Habit.—Determinate

Fragrance.—None

Fruit and seed.—None

Calyx.

Description.—16-21 inflorescence bracts, arranged symmetrically

Shape.—Linear

Apex.—Acute

Size.—Length is 1.0-1.2 cm; Width is 2.0-3.0 mm

Margin.—Entire

Color.—Upper is green (RHS 137D) and lower is green (RHS 138C)

Ray florets.

Corolla.—One petal per flower on outer whorl of flowers

Petals.—13-24

Petal size.—Length of petals is 3.0 cm; width is 0.8 cm

Petal shape.—Spatulate

Petal apex.—Obtuse with indentation at tip

Petal margin.—Entire

Petal color.—Ventral surface of ray petals is white with shades of red- purple (RHS N74C) at the tip and solid purple (RHS 77B) at the base. Dorsal surface of ray florets is purple-violet (RHS N81A) with green-yellow (RHS 1C).

Ovary.—Inferior

Style form.—One style with two stigma branches

Style color.—Yellow (RHS 4D)

Stigma color.—Purple-violet (RHS N82A)

Disk florets:

Bud shape.—Tubular

Bud size.—Length is 5 mm and width is 1 mm

Color of bud tip.—Violet-blue (RHS N92B)

Number per head.—80-85

Anther color.—Purple (RHS N77A)

Filament color.—Yellow (RHS 4D)

Pollen color.—Yellow-orange (RHS 1 7A)

### Disease and Insect Resistance

No susceptibility to diseases or insects noted to date.

### Comparison with Known Cultivars

‘Kakegawa AU7’ is most similar to the variety ‘Kakegawa AU2’, the subject of U. S. Plant patent application No. 09/657,540 which is marketed under the name ‘Sea Mist Pink and White’. The two plants differ in that ‘Kakegawa AU7’ has flower petals that are predominately white in color with a small area of bluish violet at the base of the petal surrounding the disk while the flower petals of ‘Kakegawa AU2’ are predominately pink with the basal third white in color. The following table compares the differences between the two plants.

| Characteristic | ‘Kakegawa AU7’ | ‘Kakegawa AU2’ |
|---|---|---|
| Petals | White petal with shades of | Red-purple (70B) petals |
| | red-purple (N74C) at the tip | with white (155) |
| | and purple (77B) at the base | at the base |
| Veins on | Green-yellow (1C) | Violet-purple (83B) |
| dorsal surface | | |
| of petal | | |

I claim:

1. A new and distinct cultivar of Osteospermum as shown and described herein.
